(19) United States
(12) Patent Application Publication
(10) Pub. No.: US 2004/0235190 A1
(43) Pub. Date: Nov. 25, 2004

(54) Assays for detecting, identifying and/or quantifying contaminants with liposomes

(76) Inventor: Francois Bellemare, Quebec (CA)

Correspondence Address: Goudreau Gage Dubuc, Stock Exchange Tower Suite 3400, P O Box 242, 800 Place-Victoria, Montreal, QC, H4Z 1E9, CA

(21) Appl. No.: 10/484,471

(22) PCT Filed: Jul. 23, 2002
(86) PCT No.: PCT/CA02/01139

(30) Foreign Application Priority Data
Jul. 23, 2001 (CA) 2353687

(51) Int. Cl. G01N 33/543

(52) U.S. Cl. 436/518

(57) ABSTRACT

An assay for detecting, identifying and/or quantifying at least one contaminant in a sample comprising (a) mixing the sample with liposomes having a known capture efficiency so as to form a mixture; (b) isolating the liposomes including the contaminant bound liposomes from the mixtures; (c) detecting and quantifying the captured contaminant; (d) determining the concentration of the contaminant in the sample by multiplying the liposomes' capture efficiency with the quantity of contaminant determined in (c), wherein the lipid composition of the liposomes possesses affinities with said at least one contaminant so that if the sample contains contaminants, at least a portion thereof is bound to or captured by the liposomes. The contaminants can be isolated and even concentrated in the liposomes during the assay process. The assays of the present invention are advantageously simple and inexpensive to perform and are particularly suitable for tests performed outside of sophisticated laboratories.

### FIELD OF THE INVENTION

The present invention relates to assays for isolating, concentrating, detecting and quantifying contaminants and compositions for use in same. In particular, the present invention relates to assays for isolating, concentrating, detecting and quantifying contaminants in food products, in physiological fluids including saliva, plasma, urine for veterinary and medical purposes and in fine purification processes. The present invention further relates to compositions for use in such assays.

### BACKGROUND OF THE INVENTION

The agri-food and medical industries use assays daily to detect and quantify various contaminants and other analytes in food or water and physiological fluids, respectively.

In the agri-food industry for instance, a number of bioactive molecules may participate directly or indirectly into the breeding of livestock and the growing of fruits and vegetables. For instance, antibiotics and hormones may be administered to animals directly or through their feed, and pesticides and herbicides may used to grow fruit and vegetables. Furthermore, livestock and other farm products may be affected by various infectious agents and other contaminants. It is important to detect and quantify any of those molecules in food products prior to their release in the channels of trade.

When these molecules are not detected early in the channels of trade, they may contaminate large amounts of food. For instance, milk from one herd may be mixed for shipment with that of other herds so that contaminated milk from a single cow can subsequently contaminate the milk from many herds. A simple test performed by the shipper or farmer prior to loading would enable the shipper or farmer to discard the spoiled food or keep it aside for purification treatment. Early testing could prevent important losses and avoid risks to public health.

Various methods have been proposed to detect these contaminants.

U.S. Pat. No. 4,347,312 describes an assay for detecting antibiotics in milk, meat, blood and serum comprising contacting the sample with an enzyme-labeled antibody specific to this antibiotic.

U.S. Pat. No. 5,624,850 and No. 5,976,896 describe an immunoassay for detecting analytes in liquid samples. A fluorescent ligand directed to a known analyte is added to a sample. The solution is then introduced into a capillary to which are attached ligands able to fixate the complex fluorescent ligand/analyte, if any.

U.S. Pat. No. 5,985,675 describes a immunochromatography assay. The sample is placed on the margin of a strip of material able to diffuse liquids (e.g. cellulose). Ligands specific for the analyte to detect are placed on the strip. The binding of the analyte on the ligand triggers a calorimetric reaction easy to detect.

There is also an always increasing need in the medical and veterinary industries for simpler and less costly assays to detect various analytes in physiological fluids.

Methods for detecting and quantifying analytes in physiological samples using liposomes have been proposed. International publications WO 00/72019 and WO 00/26674 disclose methods which necessitate the coupling of appropriate ligands to the liposomes' membranes to bind specific analytes/contaminants. These methods are intended for sophisticated medical laboratory purposes: the time, level of skill and type of equipment required for the preparation of ligands coupled liposomes make these methods more expensive and therefore inadequate for routine testing by less sophisticated laboratories.

Thus, there remains a need for a fast, inexpensive and reliable test for detecting contaminants in food products and physiological fluids which can be routinely used by consumers like farm, dairy plant workers, public and less sophisticated medical installations, respectively. As noted, the drawbacks of prior art assays have their relative complexity, their cost and requirement for expensive equipment.

The present invention seeks to meet these and other needs.

The present description refers to a number of documents, the content of which is herein incorporated by reference.

### SUMMARY OF THE INVENTION

The invention concerns assays for detecting and quantifying and/or isolate contaminants in food products which uses liposomes having specific membrane compositions able to bind to specific contaminants via various interactions including van der walls, hydrophilic, hydrophobic or ionic interactions and in some cases able to form complexes with contaminants.

The invention also relates to liposomal compositions for use in these assays. The lipid composition of the liposomes used for quantifying a specific analyte in a sample is selected for its ability to bind to this specific molecule. The liposomal compositions produced in accordance with the invention may have an advantageously constant capture efficiency. Once their capture efficiency is known, the concentration of the analyte/contaminant targeted by the liposomes can be determined simply by multiplying the quantity of analyte collected and the known capture efficiency rate of the liposomes used.

The liposomal compositions of the present invention can easily be prepared on a large scale. They may be advantageously stable. Furthermore, they can be advantageously prepared in large quantity and freeze-dried for later use. They can also be preserved in solution. The analytes may then be detected by a variety of assays such as HPLC, colorimetric assays, fluorescent assays, ELISA assays, immunoassays, etc. If the liposomes interfere with the assays, they can be dissolved so as to release analytes bound thereto. They are soluble in polar and non polar organic solutions and may be dissolved by detergents. In methods of the prior art using ligand coupled liposomes, the analytes are captured by the ligands. It is more difficult to detach the analytes from ligands than it is from liposome membranes. Ligands may hinder the detection of analytes bound thereto. The liposomes further are advantageously biodegradable and non toxic. They are stable in pH between 3 and 11, and saline concentrations of up to 1M. They are also able to withstand high pressure.

The invention in addition relates to kits for isolating, concentrating, detecting and quantifying contaminants in food products such as animal products including meat and milk, fruits and vegetables, food products aqueous extracts potable water and physiological fluids.

The applicant was the first to use liposomes to detect contaminants in food product samples.

The applicant was also the first to use the liposomes membrane compositions themselves to bind analytes other than antibodies specific to the various lipids composing the liposomes.

It was not predictable that specific lipidic compositions would bind specifically to certain contaminants through their lipid components.

In accordance with the present invention, there is therefore provided an assay for detecting and quantifying at least one contaminant in a sample comprising mixing the sample with liposomes having a known capture efficiency so as to form a mixture; isolating the liposomes including the contaminant bound liposomes from the mixture; detecting and metering the at least one contaminant; determining the concentration of the at least one contaminant in the sample by multiplying the liposomes' capture efficiency with the quantity of the at least one contaminant determined in (c), wherein the lipid composition of the liposomes possesses affinities with said at least one contaminant so that if the sample contains contaminants, at least a portion of the at least one contaminant is bound to the liposomes. The sample may preferably be a food sample. The detection may be performed on free contaminants or on liposomes bound contaminants.

In accordance with the present invention, there is also provided a kit for detecting and quantifying at least one contaminant in a sample comprising liposomes, liposomes isolation means and at least one detection agent for detecting the at least one contaminant.

In cases where proteins or peptides in samples are found to hinder the binding of analytes to liposomes, prior to mixing liposomes with the samples, the latter may be treated with trichloroacetic, citric or acetic acid for instance which may denature proteins, and then centrifuged to separate the supernatant from the denatured proteins.

As used herein, the designations “lipids” and “lipids compositions” is meant to include lipids per se, phospholipids, glycolipids and members of these classes or derivatives thereof including galactolipids, gangliosides, sphingosides, ceramides, sphingomyelins, sterols and any combination thereof.

As used herein, the designation “liposome” is meant to refer to lipid aqueous dispersions including small and large unilamellar vesicles, small and large multilamellar vesicles.

The terms “food product” are intended to include but are not limited to animal products such as milk, meat, blood, serum, saliva, urine; fruits and vegetables; potable water.

The terms veterinary and medical purposes are intended to include but are not limited to diagnostic and research assays to isolate, concentrate, detect and quantify analytes in physiological and biological fluids of any origin.

As used herein, the terms “contaminants” and “analytes” are used interchangeably and are meant to designate any molecule and/or bioactive substance that the methods of the present invention seeks to isolate, concentrate, detect and quantify in a sample. They include but are not limited to antibiotics, steroids, viruses, toxins, mycotoxins, hormones, peptides, herbicides, pesticides, pollutants, endocrine disrupters and nucleic acids.

The present invention also relates to a kit for detecting and quantifying contaminants in solutions of food products or of physiological fluids for veterinary and medical purposes comprising liposomes, a buffer for the liposomes and labelled ligands such as antibodies directed to contaminants, in accordance with the present invention. If the sample contains more than one analyte, different labels (i.e. fluorescent molecules such as rhodamine, fluorescein isothiocyanate (FITC), or colorimetric molecules such as HRP, alkaline phosphatase), luminescent molecules such as luminol, luciferase, etc.) can be attached to the different antibodies in order to ensure easy detection of each analyte. A compartmentalised kit in accordance with the one embodiment of the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample (e.g. milk), a container which contains the liposomes used in the assay, containers which contain wash reagents, and containers which contain the reagents used to detect or identify the complexes liposomes-contaminants.

While the methods and compositions of the instant invention are demonstrated with various types of antibiotics, herbicides, hormones, toxin and pesticides, it will be understood by the person of ordinary skill, that any contaminant having specific affinities to lipid compositions may be detected in a sample by the methods of the present invention and appropriate lipid compositions for use in these methods can be created.

Similarly, while the methods and compositions of the instant invention are demonstrated with contaminants in water and animal products, it will be apparent to one skilled in the art that these methods and compositions may be used to isolate, concentrate, detect and quantify contaminants in products other than animal derived. For instance, they can be used to detect contaminants in fruits and vegetables products as well as physiological products.

Thus according to a further feature of the present invention there are provided methods and compositions for detecting and quantifying contaminants in water.

### BRIEF DESCRIPTION OF THE DRAWINGS

Having thus generally described the invention, reference will now be made to the accompanying drawing, showing by way of illustration a preferred embodiment thereof, and in which:

FIG. 1 shows a graph illustrating how the capture efficiency of liposomes may be determined;

Other objects, advantages and features of the present invention will become more apparent upon reading of the following non-restrictive description of preferred embodiments with reference to the accompanying drawing which is exemplary and should not be interpreted as limiting the scope of the present invention.

### DESCRIPTION OF THE PREFERRED EMBODIMENT

General Protocol of Preparation of Liposomes

The nature and proportions of phospholipids, lipids and gangliosides forming the liposomal compositions are chosen in accordance with the affinities of the contaminant to detect.

Twenty milligrams of the chosen phospholipids, lipids and gangliosides are dissolved in a suitable solvent and preferably in a mixture of chloroform: methanol (2:1). Polar and non polar solvents may be used in the preparation of liposomal compositions of the present invention. Phospholipids that may be used according to the present invention include but are not limited to phosphatidylcholine (PC), phosphatidylglycerol (PG), phosphatidylserine (PS), phosphatidylethanolamine (PE), phosphatidylinositol (PI) and cardiolipine (CAR). To limit fusion of liposomes caused by certain divalent cations such as Ca⁺ and Mg⁺, one may preferably limit the use of lipids such as PS and PE and phosphatidic acid. EDTA, which binds to divalent cations, can also advantageously be added in the liposome buffer to avoid fusion. Lipids per se that may be used according to the present invention include but are not limited to cholesterol (CHOL). Glycolipids that may be used according to the present invention include but are not limited to gangliosides.

After having mixed the various lipids in suitable solvents so that the lipids are solubilized, the solvents are evaporated. Any suitable means of evaporating solvents is contemplated by the present invention. Solvents may for instance be evaporated under an inert gas stream or with a rotovapor.

The lipid mixture is then diluted in a NaCL buffer (phosphate, carbonate or citrate buffer for instance may be used), which maintains the cohesion and stability of liposomes. The dilution preferably does not exceed 10 mg/mL so as to obtain a solution that may easily be filtered and without requiring excessive pressure.

Optimum dispersion may be achieved by breaking multilamelles by sonication at a temperature above the transition temperature of the lipids aliphatic chains. Sonication may be performed in a ultrasonication bath. Alternatively, successive freezing and thawing can be performed to achieve the desired lipid dispersion.

Unilamellar vesicles can then be formed by forcing the multilamelles through an osmotic membrane. Generally, 10 runs though a 1,2 μm filtration membrane are sufficient to reach the desired result. The liposomes obtained by specific embodiments of this invention are greater than 1 μm in diameter.

For further purification, the disperson may then be run through a chromatography column such as Sepharose CL6B™ to purify them. Only descending fractions of the elution peak are preferably collected in order to obtain homogenous liposomes. Liposomes may then be at low temperature.

Although the above-described method is preferred, other methods of forming liposomes can be used to form liposomes in accordance with the present invention.

Capture Assay General Protocol

The samples to analyse may be naturally liquid or solid. In the latter case, they are diluted in water or any other appropriate buffer so as to obtain a liquid sample. Liquid samples may also be desirably diluted if the analyte is very concentrated in the sample.

An appropriate quantity of liposomal preparation is measured and mixed with the sample solution (e.g. 500 μL of liposomal preparation 2 mg/mL mixed with 5 mL of sample solution). The mixture is then stirred five minutes.

The resulting mixture is then filtered on a on a 0,45 μm filtration membrane fixed on a filter holder. Although a 0,2 μm filtration membrane was also used effectively, the 0,45 μm is preferred. Any membrane with pores of a size preventing the liposomes crossing the membrane may be used in accordance with the present. Liposomes according to specific embodiments of the present invention are greater than 1 μm in diameter. Liposomes are then collected from the filter holder with a syringe coupled with a collecting tube. Any isolation means able to isolate liposomes from the remainder of the solution can be used at this stage in accordance with the present invention. Separation techniques such as Sephadex™ or Sepharose™ column gel filtration or dialysis for instance are also appropriate means for isolating liposomes be used in accordance with the present invention. The filtration membrane is preferred because of its speed.

The captured analyte can then be metered with any suitable method. Suitable methods include but are not limited to conventional fluorescent or calorimetric assays, HPLC assays or ELISA assays. If the liposomes interfere with the assays, they can be dissolved in Triton X-100 0,03% or in an organic solvent prior to dosage. In the lipid concentrations of the specific embodiments detailed herein (less than 1 μm/mL of lipids), no interference was detected.

The present invention is illustrated in further detail by the following non-limiting example.

### EXAMPLE 1

### Preparation of PC: Chol Liposomes

Fifteen micrograms of Phosphatidylcholine (PC) (Sigma, P-3556) were taken out of the freezer and weighed on weigh paper with an analytical balance (Metler Toledo™ AB 204). The PC supply was covered with Parafilm™ immediately after usage and replaced in the freezer. The same procedure was followed to obtain 5,0 mg of cholesterol (Chol)>98% pure (# 700 000, Avanti Polar lipids™).

Lipids were transferred in a 50 mL evaporation flask and covered with aluminium foil. Five milliliters of chloroform (Fisher, C-606-1) were added to the flask. The mixture was stirred until the lipids were solubilized. A mixture PC: CHOL in a ratio 3:1, w:w is thus obtained.

The lipid mixture was then transferred in a rotovapor (BUCHI™ R-124, BUCHI waterbath™ B481, C 176) during 10 minutes at 100 rpm at 30° C. to evaporate the chloroform. Ten milliliters of phosphate buffer (0,1 M+NaCl 0,1 M pH 4,0) (carbonate or citrate buffer can also be used) was added to the mixture which was then placed for 10 minutes in an ultrasound bath (Branson™ 5210) at 38° C. The mixture was stirred regularly during sonication.

The content of the flask was transferred in a 20 mL syringe (Luer-lock™) attached to a filter holder (Swinnex™ (cat. No. SX0002500)) holding a 1,2 μm filtration membrane (MicronSep™, Cellulosic, 25 mm (cat. No. E12WP02500)). The filter holder was placed on a 15 mL Falcon™ tube. The liquid was then pushed in the tube through the membrane. The syringe was removed and the filter holder set up on a second 15 mL Falcon™ tube. The syringe was then reattached on the filter holder. The liquid of the first Falcon™ tube was transferred in the syringe for a second filtration. This procedure was repeated 8 times.

The volume of the filtered solution was adjusted with the phosphate buffer (0,1 M pH 4+NaCl 0,1 M) so as to obtain a final volume of 10 mL. The liposomes were stored in a cooler.

The level of phosphate (phospholipids) in samples was monitored in citrate or carbonate buffer. An acid digestion followed by a peroxyde oxidation was achieved on the samples before using the phosphomolybdate complex formation and reduction procedure. The 5 samples tested had an average of 60±4 μg/mL of phosphate. This may be translated into 1,4 mg/mL of phospholipids. This corresponds to a recovery higher than 90%.

### Capture of Contaminants

Standard and capture solutions are prepared as follows:

Standard # 1:1 ml of liposomes (PC: Chol; 3:1, 2 mg/mL prepared in bicarbonate, citrate or phosphate buffer 0,1 M+NaCl 0,1 M pH 4,4) and 4 ml of distilled water.

Standard # 2: Id.

Standard # 3: Id.

Capture #1: 1 mL of liposomes (PC: Chol; 3:1, 2 mg/mL prepared in bicarbonate, citrate or phosphate buffer 0,1 M+NaCl 0,1 M pH 4,4) and 4 ml of gentamycin at 0,5 μg/mL.

Capture #2: Id.

Capture #3: Id.

The capture solutions 1 to 3 were stirred 5 minutes. 6 filter holders were prepared by fixing on each a 0,22 μm or 0,45 μm filtration membrane (Osmonics).

Five millilitres of bicarbonate, citrate or phosphate buffer (0,1M+NaCl 0,1M pH 4,4) were pushed with 5 mL syringes through the filtration membranes.

The standard and capture solutions were pushed through separate filter holders a 20 mL syringe fixed to each filter holders.

### Immunodetection of Contaminants

The undersides of the filter holders were sealed with paraffin to avoid leakage and the filter holders were filled with 1.3 mL of anti-gentamycin antibodies 1:10 000 (dilution in PBS-Tween™ 0,5%) with a Pasteur pipette.

The filter holders were incubated 10 minutes at 37° C. The parafin seals were withdrawn and the filters were then emptied by air pressure with a 5 mL syringe. 5 mL of PBS-Tween™ 0,5% were pushed through the filtration membranes with a 5 mL syringe to release non fixed antibodies.

The undersides of the filter holders were again sealed with paraffin and each filled with 1,3 mL of anti-IgG labelled with horseradish peroxydase 1:750 (dilution in PBS-Tween™ 0,5%) with a Pasteur pipette.

Filter holders and their content were then incubated 10 minutes at 37° C. They were then emptied again by air pressure with a 5 mL syringe. Five millilitres of PBS-Tween™ 0,5% were then pushed through the filtration membranes with a 5 mL syringe to release non fixed antibodies.

The undersides of the filter holders were again sealed with paraffin and then filled with 1 ml of phosphate-citrate buffer 0,1 M pH 4. The filter holders are then shaken vigorously.

Pasteur pipettes or 5 mL syringes were used to recover the content of the filter holder. A syringe could also be used to draw the content up and by turning the filter holder over. 100 μl of the content of each filter holder was then transferred in separate wells of a 96 wells ELISA plate. 200 μL of substrate (2,2′, azinodi (3-ethyl) benzo thiazolinei-6-sulfonic acid) was then added to each well. The plate was placed away from light for color development.

### Results

Absorbency readings of the ELISA plate of Example 3 are taken at 405 nm after 15 minutes and after 17 hours.

TABLE 1
ELISA measurement of capture efficiency of liposomes
measured after 17 hours

| Standard | Standard | Standard | Capture | Capture | Capture |
|---|---|---|---|---|---|
| # 1 | # 2 | # 3 | # 1 | # 2 | # 3 |
| 0.081 | 0.185 | 0.164 | 0.446 | 0.255 | 0.576 |
| 0.083 | 0.189 | 0.148 | 0.372 | 0.213 | 0.431 |
| 0.078 | 0.184 | 0.152 | 0.384 | 0.248 | 0.367 |
| — | 0.175 | — | 0.364 | — | — |

| | Stan- | Standard | Standard | Capture | Capture | Capture |
|---|---|---|---|---|---|---|
| | dard # 1 | # 2 | # 3 | # 1 | # 2 | # 3 |
| Average | 0.0807 | 0.1833 | 0.1547 | 0.3915 | 0.2387 | 0.4580 |
| Standard | 0.0025 | 0.0059 | 0.0083 | 0.0373 | 0.0225 | 0.1071 |
| deviation | | | | | | |

TABLE 2
ELISA measurement of capture efficiency of liposomes
measured after 15 minutes

| Standard | Standard | Standard | Capture | Capture | Capture |
|---|---|---|---|---|---|
| # 1 | # 2 | # 3 | # 1 | # 2 | # 3 |
| 0.055 | 0.076 | 0.075 | 0.132 | 0.092 | 0.154 |
| 0.054 | 0.074 | 0.066 | 0.108 | 0.082 | 0.122 |
| 0.053 | 0.071 | 0.066 | 0.110 | 0.086 | 0.110 |
| | 0.070 | | 0.108 | | |

| | Stan- | Standard | Standard | Capture | Capture | Capture |
|---|---|---|---|---|---|---|
| | dard # 1 | # 2 | # 3 | # 1 | # 2 | # 3 |
| Average | 0.0540 | 0.0728 | 0.069 | 0.1145 | 0.0867 | 0.1287 |
| Standard | 0.0010 | 0.0028 | 0.0052 | 0.0117 | 0.0050 | 0.0227 |
| deviation | | | | | | |

Table 3 below presents capture efficiencies, as determined by methods similar to those disclosed in above-presented examples, of various liposomal compositions, prepared according to the present invention, for various types of antibiotics namely aminoglycosides (gentamycin and neomycin), a beta-lactamase (penicillin) and a sulfamide (sulfamethazine) and a herbicide (atrazine).

TABLE 3
Capture efficiency of various liposomal compositions with
various analytes

| | | CAPTURE |
|---|---|---|
| | | EFFICIENCY |
| | | (AVERAGE ± |
| LIPOSOME | | STANDARD |
| COMPOSITION | CAPTURE SOLUTION | DEVIATION) |
| A₁ (PC:PG; 2:1) | 0.5 mg of liposome for 1 ml of | 64.2% and 56.6% |
| | sulfamethazine 0.2 μg/ml | (60.4 ± 5.4) |
| A₂ (PC:PG; 1.5:1) | 0.5 mg of liposome for 1 ml of | 49.2%; 45.4%; |
| | sulfamethazine 0.2 μg/ml | 42.6% and 57.4% |
| | | (48.6 ± 6.4) |
| A₃ (PC:PG; 3:1) | 0.5 mg of liposome for 1 ml of | 59.5%; 58.3% and |
| | sulfamethazine 0.2 μg/ml | 77.9% |
| | | (65.2 ± 11.0) |
| A₅ (PC:PG; 6.5:1) | 0.5 mg of liposome for 1 ml of | 64.2%; 60.5%; |
| | sulfamethazine 0.2 μg/ml | 52.9%; 61.7%; |
| | | 61.7%; 69.9%; |
| | | 64.2%; 54.8% |
| | | and 54.8% |
| | | (60.5 ± 5.4) |
| A₆ (PC:PG; 7:1) | 0.5 mg of liposome for 1 ml of | 70.8% and 89.8% |
| | sulfamethazine 0.2 μg/ml | (80.3 ± 13.4) |
| A₄ (PC:PG; 5:1) | 0.2 mg of liposome for 5 ml of | 16.5% and 13.0% |
| | penicillin 0.2 μg/ml | (14.75 ± 2.5) |
| | 0.5 mg of liposome for 5 ml of | 27.0% and 43% |
| | penicillin 0.2 μg/ml | (35 ± 11.3) |
| A₄ (PC:PG; 5:1) | 0.26 mg of liposome for 4 ml | 4% |
| | of gentamycin 1.0 μg/ml | |
| | 0.53 mg of liposome for 4 ml | 12% |
| | of gentamycin 1.0 μg/ml | |
| | 1.32 mg of liposome for 4 ml | 37% |
| | of gentamycin 1.0 μg/ml | |
| | 2.64 mg of liposome for 4 ml | 50% |
| | of gentamycin 1.0 μg/ml | |
| A₇ (PC:PG; 10:1) | 0.055 mg of liposome for 4 ml | 26.2 |
| | of gentamycin 1.0 μg/ml | |
| | 0.11 mg of liposome for 4 ml | 28.8% |
| | of gentamycin 1.0 μg/ml | |
| | 0.28 mg of liposome for 4 ml | 47.0% |
| | of gentamycin 1.0 μg/ml | |
| | 0.55 mg of liposome for 4 ml | 61.1% |
| | of gentamycin 1.0 μg/ml | |
| F (PC:Chol; 2:1) | 0.060 mg of liposome for 4 ml | 45.0% |
| | of gentamycin 1.0 μg/ml | |
| | 0.12 mg of liposome for 4 ml | 54.0% |
| | gentamycin 1.0 μg/ml | |
| | 0.30 mg of liposome for 4 ml | 72.5% |
| | gentamycin 1.0 μg/ml | |
| | 0.60 mg of liposome for 4 ml | 77.0 |
| | of gentamycin 1.0 μg/ml | |
| | 1 mg of liposome for 5 ml of | 90.0% and 90.0% |
| | gentamycin 1 μg/ml | (90 ± 0) |
| | 1 mg of liposome for 6 ml of | 12.0%; 12.8% |
| | gentamycin 0.5 μg/ml | 20.0% and 21.2% |
| | | (16.5 ± 4.8) |
| | 1 mg of liposome for 5 ml of | 27.4% |
| | gentamycin 0.5 μg/ml | |
| H (PC:Chol; 1:1) | 1 mg of liposome for 6 ml of | 56.8%, 57.0% and |
| | gentamycin 0.5 μg/ml | 53% |
| | | (55.6 ± 2.3) |
| I (PC:Chol; 3:1) | 0.50 mg of liposome for 6 ml | 32.3% |
| | of gentamycin 0.5 μg/ml | |
| | 1.0 mg of liposome for 6 ml of | 70.8%; 70.7% and |
| | gentamycin 0.5 μg/ml | 65.0% |
| | | (68.8 ± 3.3) |
| | 1.0 mg of liposome for 5 ml of | 100% and 85% |
| | gentamycin 1 μg/ml | (92.5 ± 10.6) |
| | 2.0 mg of liposome for 6 ml of | 100% and 98% |
| | gentamycin 0.5 μg/ml | (99 ± 1.4) |
| | 4.0 mg of liposome for 6 ml of | 99.3% and 92.0% |
| | gentamycin 0.5 μg/ml | (95.6 ± 5.2) |
| I (PC:Chol; 3:1) | 0.10 mg of liposome for 4 ml | 34.4% |
| | of neomycin 1.5 μg/ml | |
| | 0.2 mg of liposome for 4 ml of | 51.8% |
| | neomycin 1.5 μg/ml | |
| | 0.5 mg of liposome for 4 ml of | 75.8% |
| | neomycin 1.5 μg/ml | |
| | 1.0 mg of liposome for 4 ml of | 100% |
| | neomycin 1.5 μg/ml | |
| | 2.0 mg of liposome for 4 ml of | 100% |
| | neomycin 1.5 μg/ml | |
| A₄ (PC:PG; 5:1) | 0.86 mg of liposome for 5 ml | 85.0% |
| | of atrazine 10 μg/ml | |

Capture efficiencies of these various liposomal compositions are shown to be significant and stable from one assay to the other.

Various other capture solutions were prepared and tested according to the procedure described in Example 1 under the title Capture of contaminants and Table 4 indicates which of the liposomal compositions tested achieved the best capture efficiency with analytes tested including antibiotics, hormones, a herbicide, a pesticide and a toxin. The capture efficiency was measured after 5 minutes of incubation of a capture solution containing one mg of the liposomal composition and 1 μg of the bioactive molecule. Of note, for the cholera toxin, the pH of the solution was 5, and the effective ratio liposome/protein was 4 mg/μg.

TABLE 4
Summary of capture efficiencies of specific liposomes
types with various representative analytes

| LIPOSOME TYPE | I | A₇ | LL | MER 3 | MH | MER 2 |
|---|---|---|---|---|---|---|
| Gentamicin/ | 100% | | | | | |
| Neomycin | | | | | | |
| Atrazine | | 75% | | | | |
| Sulfamethazine | | 85% | | | | |
| 2,4-D | | | 100% | | | |
| Penicillin | | | | 100% | | |
| Progesterone/ | | | | | 94% | |
| Estradiol | | | | | | |
| Tetracycline | | | | | | 85% |
| Cholera | | | | | | 100% |

Legend:
Type I: PC:CHOL (3:1)
Type A₇: PC:PG (10:1)
Type Mer 3: PC:PG:PE (10:10:5)
Type LL: PC:PE (10:1)
Type MH: PC
Type Mer 2: PC:PE:PG:CHOL (50:25:20:5)

The type of filtration membrane used may affect the measured capture efficiency of the assays depending on the analyte captured. It should therefore be chosen accordingly. For instance, membranes constituted of mixed ester of cellulose adsorb the progesterone and estradiol hormones, but not the antibiotics or the cholera toxin. Polycarbonate membranes are thus more appropriate to capture hormones. Larger volumes can be processed using a peristaltic pump instead of a syringe.

The recovery of the liposomes from the filtration apparatus is very important when they are used to isolate and concentrate analytes. The best recovery was obtained where a detergent was used to detach the adsorbed liposomes from the filtration membrane. A Triton solution of 1% allowed the recovery of more than 80% of the filtered liposomes. The bioactive molecules were then available for standard analysis assays including HPLC, ELISA.

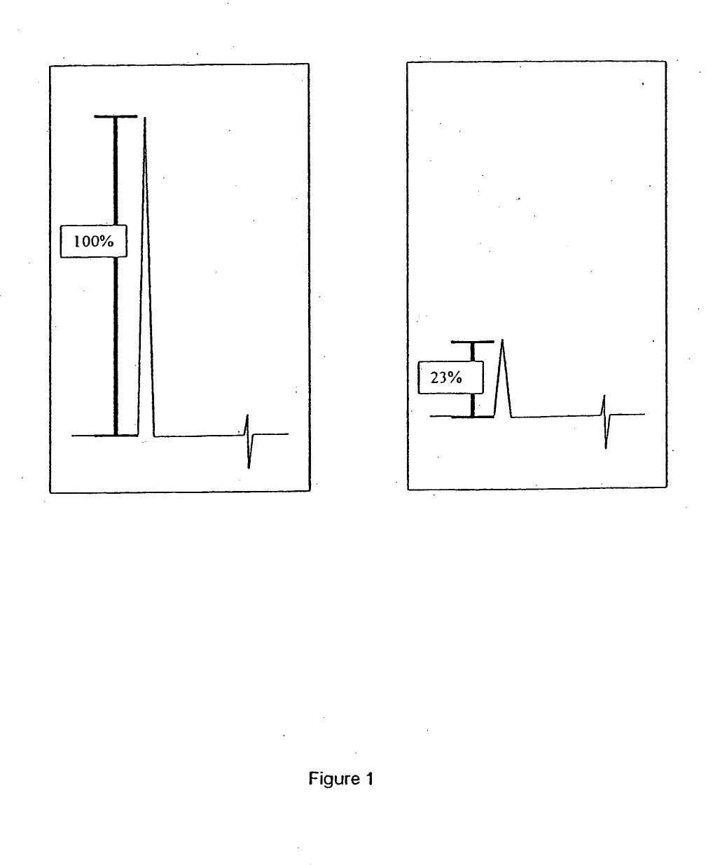

As is illustrated in FIG. 1, the capture efficiency of the various liposomes may be determined by comparing the signal of a known contaminant in a solution with that of the filtrate of the solution having been mixed with the liposomal preparation and filtered. Hence as illustrated in FIG. 1, the signal produced by a contaminant in a sample is compared with that produced by the contaminant in the filtered solution. The filtered sample signal is given a value relative to that of the sample solution prior filtration. In FIG. 1, the filtered sample produces a signal which corresponds to 23% of that produced by the sample prior treatment. It can therefore be deduced that 77% of the contaminants were captured by the liposomes so that the capture efficiency of these liposomes is determined to be 77%.

Underlying the above results, certain principles in lipids-analytes affinities are apparent that may help selecting lipid compositions suitable for the methods of the present invention. Hence in general liposomes comprising a neutral phospholipid (phosphatidylcholine) as a major component and a negatively charged phospholipid (phosphatidyl glycerol) or a neutral one (cholesterol) as a minor component are capable of capturing many analytes having an amino moiety. The capture efficiency is generally directly dependent on the relative quantity of total lipids with regard to the quantity of analytes, or on the relative quantity of the major lipid component in the lipid mixture. The hydrophilicity or hydrophobicity can be modified to suit the nature of the analyte by acylation of the phospholipids.

Interactions between analytes and cells are also good indications permitting one skilled in the art to determine suitable lipid compositions for the methods and compositions of the present invention. Hence, aminoglycosides need to penetrate cells membranes to act: a PC: CHOL mixture is then suitable to capture these antibiotics.

Herbicides act on plant cells containing PC and PG so that a liposome constituted of these lipids is efficient to capture herbicides. Viruses often bind the cells membrane before infection and bind more particularly to gangliosides in cells membranes. It is expected that a mixture of PC: gangliosides or PE: gangliosides will be efficient to produce liposomes able to capture viruses.

Toxins mechanism of action comprises creating pores in cells membranes. PE is known to create pores in acid media so that a mixture of PC:PE is expected to produce liposomes that can efficiently bind toxins.

Although the present invention has been described hereinabove by way of preferred embodiments thereof, it can be modified, without departing from the spirit and nature of the subject invention as defined in the appended claims.

1. Assay for detecting, identifying and/or quantifying a contaminant which has an affinity for liposomes lipid constituents, in a sample comprising (a) mixing the sample with liposomes having a known capture efficiency towards contaminant so as to form a mixture which includes contaminant bound liposomes; (b) isolating the contaminant bound liposomes from the mixture; (c) detecting the presence and the quantity of the contaminant in said contaminant bound liposomes; and; (d) quantifying the contaminant in the sample by multiplying the liposomes' capture efficiency with the quantity of contaminant in the contaminant bound liposomes.

2. Assay as in claim 1 wherein said contaminant is selected from the group consisting of antibiotics, steroids, viruses, mycotoxins, toxins, hormones, peptides, herbicides, pesticides, pollutants, endocrine disrupters and nucleic acids.

3. An assay as in claim 1 wherein the lipid constituents of the liposomes include phosphatidylcholine.

4. An assay as in claim 3 wherein the contaminant is an aminoglycoside and the lipid constituents are phosphatidylcholine: cholesterol in a ratio of 3:1.

5. An assay as in claim 3 wherein the contaminant is 2,4-Dinitrophenol and the lipid constituents are phosphatidylcholine: phosphatidylethanolamine in a ratio of 1 0:1.-23

6. An assay as in claim 3 wherein the contaminant is penicillin and the lipid constituents are phosphatidylcholine:phosphatidylglycerol:phosphatidylethanolamine in a ratio of 10:10:5.

7. An assay as in claim 3 wherein the contaminant is selected from the group consisting of a cholera toxin and tetracycline and the lipid constituents are phosphatidylcholine: phosphatidylethanolamine: phosphatidylglycerol: cholesterol in a ratio of 50:25:20:5.

8. An assay as in claim 3 wherein the contaminant is an hormone and the lipid constituent is phosphatidylcholine.

9. An assay as in claim 8 wherein the hormone is selected from the group consisting of progesterone and estradiol.

10. An assay as in claim 3 wherein the contaminant is selected from the group consisting of sulfamethazine and atrazine and the lipid constituents are phosphatidyl choline: phosphatidylglycerol in a ratio of 1 0:1.

11. An assay as in claim 1 wherein the sample is selected from the group consisting of food products, aqueous extracts of food products, water and physiological fluids.

12. An assay as in claim 1 wherein the known capture efficiency of the liposomes is about 1 00%.

13. An assay as in claim 1 wherein the step c) of detecting the contaminant is performed directly on liposomes-bound contaminants.

14. A kit for detecting, identifying and/or quantifying at least one contaminant in a sample comprising
(a) liposomes;
(b) contaminant bound liposomes isolation means;
(c) at least one detection agent for detecting, identifying and/or quantifying the contaminant present on the contaminant bound liposomes.

15. A kit as in claim 14 wherein the liposomes comprise phosphatidylcholine.

16. A method of selecting the lipid composition of a liposome for use in the capture of a contaminant comprising: (a) producing a liposome having a specific lipid composition comprising phosphatidylcholine; (b) determining the capture efficiency of the liposome on the contaminant, wherein the lipid composition is selected if the capture efficiency determined in (b) is significant and stable.
